(19) United States
(12) Patent Application Publication
(10) Pub. No.: US 2016/0131650 A1
(43) Pub. Date: May 12, 2016

(54) VIRUS-DETECTION KITS AND METHODS OF MAKING AND USING THE SAME

(71) Applicant: EMORY UNIVERSITY, Atlanta, GA (US)

(72) Inventors: Rostam Zafari, Decatur, GA (US); Brian James Goldstone, Corona del Mar, CA (US); Justine Suzanne Liepkalns, Atlanta, GA (US)

(21) Appl. No.: 14/873,813

(22) Filed: Oct. 2, 2015

Related U.S. Application Data
(60) Provisional application No. 62/059,834, filed on Oct. 3, 2014.

(51) Int. Cl. G01N 33/569; G01N 33/543

(52) U.S. Cl. CPC G01N 33/56983; G01N 33/54366; G01N 2333/08

(57) ABSTRACT

The present disclosure relates to compositions, kits, and methods for the detection of a virus in a sample. Typically, the sample is from a subject suspected of, exhibiting symptoms of, or previously diagnosed with a viral infection, such as an Ebola virus.

### CROSS REFERENCE TO RELATED APPLICATIONS

This Application claims priority to U.S. Provisional Application No. 62/059,834 filed Oct. 3, 2014, hereby incorporated by reference in its entirety.

### BACKGROUND

The Ebola virus, an RNA virus stemming from the family Filoviridae, is the root cause of Ebola virus disease (EVD). This virus is found in remote villages with tropical climates near rainforests. It has five known strains, three of which are commonly culpable for large outbreaks of EVD in Africa. These three strains include Bundibugyo ebolavirus (BDBV), Zaire ebolavirus (EBOV), and Sudan ebolavirus (SUDV). With regards to EVD's rapid human-to-human transmission, the main vehicles for transmission are exchanges of bodily fluids (e.g., blood, saliva, and mucous) through direct contact. The symptoms of EVD include, but are not limited to, sudden onset of fever, intense weakness, muscle pain, headache and sore throat, and as the illness progresses, vomiting, diarrhea, rash, impaired liver and kidney function, followed by external and internal hemorrhage.

The early symptoms resemble those resulting from other illness and therefore proper care is not administered. Since the Ebola is contagious when symptoms arise, others who come in contact with the patient are now at risk. As the patient symptoms progress to hemorrhaging, their life is therefore at risk of excessive blood loss, which is how patients succumb to EVD. Detecting this disease when before or during early symptoms (often overlooked) will not only inform the patient to seek proper care but also limit transmission to others (particularly regarding travelers). Previously, EVD stayed in east Africa where the population has already begun to co-evolve, however this virus has increased is incubation period (the time between infection and the appearance of the first symptoms) to 21 days. Naïve populations are now exposed to this virus and are ill prepared for its effects.

Current methods for Ebola detection include Polymerase Chain Reaction (PCR), which can run assays for a relatively small cost. However, the start-up price of the machinery costs anywhere from $10,000 to over $100,000. Since Ebola is mostly concentrated in areas of a low socio-economic climate, these costs are significant obstacles for detection of the virus. More urgently, these machines cement the process of detection to the hospital, as they are not meant for mobility and require electricity. Detection of serum anti-Ebola antibodies in a patient can also be disadvantageous since the progression of EVD can be more rapid than the development of the patient's antibodies against the Ebola virus.

The World Health Organization (WHO) highlights that outbreaks of EVD are initially concentrated in remote villages of Central and Western Africa. Many people infected with Ebola die before reaching hospitals for treatment, with potentially more that may refuse or are unable to reach a hospital.

According to the WHO, the incubation period for the Ebola virus ranges from 2 to 21 days and has a case mortality rate of up to 90%. This long incubation period allows the virus to travel far undetected and cause death in particularly susceptible populations that are naïve to this virus. As the CDC reported in October 2014, the number of EVD infected people in Sierra Leone and Liberia is predicted to double every 20 days.

Thus, there is a need to provide quick and user friendly (i) testing methods for detecting a given virus, such as the Ebola virus, and (ii) subsequent treatment of infected individuals.

### SUMMARY

The present disclosure relates to compositions, kits, and methods for the detection of a virus in a sample. Typically, the sample is from a subject suspected of, exhibiting symptoms of, or previously diagnosed with a viral infection, such as an Ebola virus.

In certain embodiments, the present disclosure provides virus-detection kits. The virus-detection kits of the present disclosure provide one or more of the following benefits: (1) ease to produce; (2) ease of use; (3) inexpensive to make and use; (4) quick detection of a given virus; and (5) mobility of detection kit/procedure so as not to restrict the location for a given test.

In certain embodiments, the kits comprise a substrate such as nitrocellulose, an antibody to a viral coat protein, a tube, and a cap comprising a substrate configured such that the substrate fits inside the tube, and optionally a second antibody that binds the antibody to the viral coat protein, wherein the second antibody is conjugated to a visual marker or other moiety having or capable of creating a light signal, e.g., fluorescent marker, gold particle, or enzyme capable of creating a fluorescent molecule in the presence of a substrate, which can be seen with the naked eye.

In certain embodiments, the disclosure relates to a Rapid Early Detection Strips (REDS) kit. The REDS kit is intended to be portable and user-friendly, allowing early virus-causing disease (e.g., EVD) detection, for example, while screening in a village setting. Additionally, the REDS kit can potentially test for EVD before symptoms occur since it captures/detects the antigen in a person's blood or saliva. Furthermore, the use of saliva avoids the use of needles and the risks of contamination that go along with it, as well as allowing individuals that refuse needle pricks to be tested. This test is portable and does not need the use of a machine, such as PCR, and allows for a relatively rapid answer.

In some exemplary embodiments of the present disclosure, the virus-detection kit comprises Rapid Early Detection Strips (REDS), which may be used to detect the presence of the Ebola virus. REDS, and the user-friendly kits containing REDS, can be designed to test for Ebola, or any other applicable virus, in a portable and inexpensive method compared with current procedures.

Accordingly, in one exemplary embodiment, the present disclosure is directed to virus-detection kits. In some exemplary embodiments of the present disclosure, the virus-detection kit comprises: one or more test strips, each test strip comprising a strip substrate; one or more reagent solutions in individual solution containers, said one or more reagent solutions comprising (i) at least one wash solution comprising a phosphate buffered saline (PBS) solution, (ii) at least one unconjugated coating antibody solution, and (iii) at least one conjugated primary antibody solution; one or more test tubes sized to contain a desired amount of a given solution; and one or more test tube caps sized to seal a given test tube.

In other exemplary embodiments of the present disclosure, the virus-detection kit comprises: one or more coated test strips, each coated test strip comprising a strip substrate and a coating thereon, said coating comprising at least one unconjugated coating antibody capable of binding with a given virus; one or more reagent solutions in individual solution containers, said one or more reagent solutions comprising (i) at least one wash solution comprising a phosphate buffered saline (PBS) solution, and (ii) at least one conjugated primary antibody solution; one or more test tubes sized to contain up to about 1000 milliliters of a given solution; and one or more test tube caps sized to seal a given test tube.

The present disclosure is further directed to methods of making virus-detection kits. In some exemplary embodiments of the present disclosure, the method of making a virus-detection kit comprises making any of the herein-described virus-detection kits.

The present disclosure is even further directed to methods of using virus-detection kits. In some exemplary embodiments of the present disclosure, the method of using a virus-detection kit comprises using any of the herein-described virus-detection kits to detect the presence of a virus, such as the Ebola virus.

In certain embodiments, the disclosure relates to methods of detecting the presence of a virus such as Ebola virus, in a sample comprising a) mixing a solid substrate such as a test strip, that has an antibody that binds a viral particle or protein immobilized to the solid substrate with a sample suspected of containing a virus under conditions such that the antibody binds the viral particle or protein providing an area of the substrate having an immobilized antibody bound viral particle or protein and b) mixing the area having a substrate immobilized antibody bound viral particle or protein with a secondary antibody that also binds a portion of the viral particle or protein under conditions such that the secondary antibody provides a signal that of the presence of the virus particle or protein in the sample.

In certain embodiments, the disclosure relates to methods of detecting the presence of a virus such as Ebola virus, in a sample comprising a) mixing a solid substrate such as a test strip and a sample suspected of containing viral particle or protein under conditions such that the viral particle or protein is immobilized to an area the solid substrate w and b) mixing the area having a substrate immobilized viral particle or protein with a primary antibody that binds a portion of the viral particle or protein under conditions such that the antibody provides a signal that of the presence of the virus particle or protein in the sample. In certain embodiments, the primary antibody provided a signal due to the binding of a secondary antibody to the constant region of the primary antibody. The detection of the virus may be performed by comparing the similarity or dis-similarity of a signal of the antibodies to a reference, standard, or normal sample, e.g., a differed in signal of a sample comprising a viral particle or protein and or not containing a virus particle or protein.

In certain embodiments, the disclosure contemplates the use of capillary action as the serum (with or without blood) or saliva undergoes the capillary action throughout the strip it can pick up on the various antibody(ies) and, if applicable, the substrate or development solution. This method would allow all that is described above to occur in a simple solution movement, without having to transfer a strip from bottle to bottle.

### BRIEF DESCRIPTION OF THE FIGURES

FIG. 1 is a perspective view of an exemplary virus-detection kit 10 of the present disclosure. On side 13 of the box 11 may contain colors that correlated to a visual marker that is used in the assay for comparison. Step-by-step instructions 16 may be on an outer surface 17.

FIG. 2 is a perspective view of exemplary contents within the exemplary virus-detection kit shown in FIG. 1. The circles 12 on opposite sides 13/14 of box 11 represent tube holders where bottles/tubes 18 will be placed in a specific and predetermined order. Since the placement of every tube 18 will be labeled with a number and the instructions 16 will be on outer surface 17 of box 11, a health-care professional simply puts each tube 18 where it is marked and follows the step-by-step instructions. A first area 21 provides space for items such as a mask, gloves, and pipettes. Individual solution containers 22 contain reagent solutions such as a wash solution comprising a phosphate buffered saline (PBS) solution, an unconjugated coating antibody solution, and a conjugated primary or secondary antibody solution. A second area 23 is also provides space for test strips 19.

FIG. 3 illustrates a tub 18 with a cap 20 with a strip 19 through it. This effective design can be used with any applicable strip test to increase portability, cost-effectiveness, user-friendliness, and minimize contamination of uninfected healthcare workers. The tubes containing specific solutions can be re-used making this process cost effective. Once exposed to the patient's serum or saliva, the strip 19 is placed in a cap and the healthcare worker now only transfers the cap from bottle to bottle, minimizing the healthcare worker's exposure to the Ebola virus if present in the sample collected.

### DETAILED DESCRIPTION

To promote an understanding of the principles of the present disclosure, descriptions of specific embodiments of the disclosure follow and specific language is used to describe the specific embodiments. It will nevertheless be understood that no limitation of the scope of the disclosure is intended by the use of specific language. Alterations, further modifications, and such further applications of the principles of the present disclosure discussed are contemplated as would normally occur to one ordinarily skilled in the art to which the disclosure pertains.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent. Prior to describing the various embodiments, the following definitions are provided and should be used unless otherwise indicated.

It should be understood that although the above-described virus-detection kits and/or methods are described as “comprising” one or more components or steps, the above-described virus-detection kits and/or methods may “comprise,” “consists of,” or “consist essentially of” the above-described components, features or steps of the virus-detection kits and/or methods. Consequently, where the present disclosure, or a portion thereof, has been described with an open-ended term such as “comprising,” it should be readily understood that (unless otherwise stated) the description of the present disclosure, or the portion thereof, should also be interpreted to describe the present disclosure, or a portion thereof, using the terms “consisting essentially of” or “consisting of” or variations thereof as discussed below.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a virus-detection kit and/or method that “comprises” a list of elements (e.g., components, features, or steps) is not necessarily limited to only those elements (or components or steps), but may include other elements (or components or steps) not expressly listed or inherent to the virus-detection kit and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a virus-detection kit and/or method that includes materials, steps, features, components, or elements, in addition to those literally disclosed, provided that these additional materials, steps, features, components, or elements do not materially affect the basic and novel characteristic(s) of the claimed disclosure. The term “consisting essentially of” occupies a middle ground between “comprising” and “consisting of”.

The terms “determining”, “measuring”, “evaluating”, “assessing,” “assaying,” and “analyzing” can be used interchangeably herein to refer to any form of measurement, and include determining if an element is present or not. These terms can include both quantitative and/or qualitative determinations. Assessing may be relative or absolute. “Assessing the presence of” can include determining the amount of something present, as well as determining whether it is present or absent.

A “blood sample” is a biological sample which is derived from blood, preferably peripheral (or circulating) blood. A blood sample may be, for example, whole blood, plasma or serum or cell therein.

An “individual” or “subject” is a mammal, more preferably a human. Mammals include, but are not limited to, humans, primates, farm animals, sport animals, rodents and pets.

A “normal” individual or sample from a “normal” individual as used herein for quantitative and qualitative data refers to an individual who has or would be assessed by a physician as not having a viral infection.

As used herein, a “reference value” can be an absolute value; a relative value; a value that has an upper and/or lower limit; a range of values; an average value; a median value, a mean value, or a value as compared to a particular control or baseline value. A reference value can be based on an individual sample value, such as for example, a value obtained from a sample from the individual with viral infection, but at an earlier point in time, or a value obtained from a sample from a viral infected patient other than the individual being tested, or a “normal” individual, that is an individual not diagnosed with viral infection. The reference value can be based on a large number of samples, such as from viral infected patients or normal individuals or based on a pool of samples including or excluding the sample to be tested.

The process of comparing a measured value and a reference value can be carried out in any convenient manner appropriate to the type of measured value and reference value. Measuring can be performed using quantitative or qualitative measurement techniques, and the mode of comparing a measured value and a reference value can vary depending on the measurement technology employed. For example, when a qualitative calorimetric assay is used to measure levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). As with qualitative measurements, the comparison can be made by inspecting the numerical data, by inspecting representations of the data (e.g., inspecting graphical representations such as bar or line graphs).

The process of comparing may be manual (such as visual inspection by the practitioner of the method) or it may be automated. For example, an assay device (such as a luminometer for measuring chemiluminescent signals) may include circuitry and software enabling it to compare a measured value with a reference value. Alternately, a separate device (e.g., a digital computer) may be used to compare the measured value(s) and the reference value(s). Automated devices for comparison may include stored reference values being measured, or they may compare the measured value(s) with reference values that are derived from contemporaneously measured reference samples.

In some embodiments, the methods of the disclosure utilize “simple” or “binary” comparison between the measured level(s) and the reference level(s) (e.g., the comparison between a measured level and a reference level determines whether the measured level is higher or lower than the reference level).

Virus-Detection Kits

The present disclosure is directed to virus-detection kits. The present disclosure is further directed to methods of making virus-detection kits. The present disclosure is even further directed to methods of using virus-detection kits.

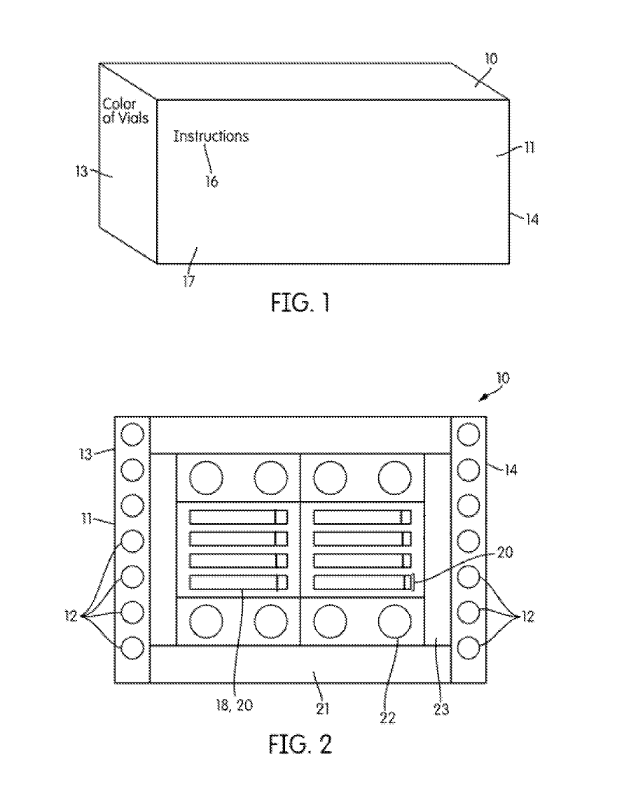

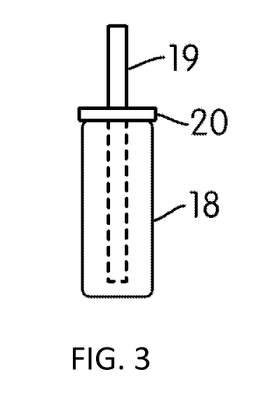

As shown in FIGS. 1-2, exemplary virus-detection kit 10 of the present disclosure may comprise components so as to be able to perform multiple tests. Exemplary virus-detection kit 10 comprises box 11 having contents that are arranged so that a health-care professional (not shown) can perform a user-friendly and portable test. The circles 12 on opposite sides 13/14 of box 11 represent tube holders where tubes/vials 18 will be placed in a specific and predetermined order. The step-by-step instructions 16 will be on an outer surface 17 of box 11. Since the placement of every tube 18 will be labeled with a number and the instructions 16 will be on outer surface 17 of box 11, a health-care professional (not shown) simply puts each tube 18 where it is marked and follows the step-by-step instructions.

A cap 20 with a strip 19 through it is transferred to the appropriate bottles 18 step-by-step (instructions will be provided 16 on time of incubation, and whether to shake/invert). This effective design can be used with any applicable strip test to increase portability, cost-effectiveness, user-friendliness, and minimize contamination of uninfected healthcare workers. The tubes containing specific solutions can be re-used making this process cost effective. Once exposed to the patient's serum or saliva, the strip 19 is placed in a cap and the healthcare worker now only transfers the cap from bottle to bottle (sealing them each time), minimizing the healthcare worker's exposure to the Ebola virus (if present in the sample collected). The bottles 18 will be numbered 1, 2, 3, etc. making the bottle transfer method relatively simple therefore minimizing human error.

The following steps may be used to prepare a given strip 19 by coating with anti-Ebola antibodies. Desirably, the strip coating step is performed prior to the formation of a given kit so that the kit contains coated test strips. However, in other embodiments, appropriate solutions and other kit components are provided within a given kit so that a user can prepare coated test strips just prior to use.

Coated test strips may be prepared using the following exemplary steps. Submerge strip 19 with enough volume necessary to cover the entire strip 19 with diluted unconjugated coating anti-Ebola antibodies. These antibodies may be, for example, Anti-Ebola virus antibody [FE18], Anti-Ebola virus antibody [FE37], and Anti-Ebola virus antibody [FE25]. These commercially available antibodies are available from a number of different medical companies including, but not limited to, Abcam and Thermo Fisher Scientific Inc.

Each strip 19 is desirably submerged in a concentration of about 5 μg of antibody per milliliter (ml) of phosphate buffered saline (PBS). This concentration may be adjusted as desired for minimal concentration and maximal detection. Incubate the strip(s) 19 with the coating antibody for 2 hr at 37° C., or overnight at 4° C. (i.e., in a refrigerator). Incubation time can be varied as desired to obtain a desired short time with optimal results.

If the coating process is one of the method steps provided in step-by-step instructions, for example, on an outer surface of the kit, this step will include first placing the strip 19 in a cap 20, then placing both on a bottle containing the above optimal concentration of unconjugated coating anti-Ebola antibodies, and after screwing tightly, inverting the bottle and cap/strip 19 and 20 for a period of time and/or leaving solution to incubate for a desired period of time.

Wash the strip(s) 19 with PBS 3 times: first remove strip(s) 19 from above coating solution, then submerge them in PBS and gently shake/swirl/invert for 3 to 5 minutes (optionally on a rocker). If the coating process is one of the method steps provided in step-by-step instructions, this wash will be by using a squeeze bottle to submerge the strip/cap 19 and 20. The time can be varied as desired to obtain a desired short time with optimal results. It is likely best to do washes by adding the wash solution onto the strip via something like a squeeze bottle in order to not contaminate the wash solution that is to be used multiple times (for multiple strips).

Blocking non-specific binding: Submerge the strip 19 in PBS solution containing 4% BSA (Bovine Serum Albumin) and incubate ≧1 hr at room temperature (optionally leave it on a rocker). To make PBS/4% BSA, add 4 g (grams) of BSA in 100 ml PBS. The concentration and time may be varied for best results and shortest time and may contain a detergent. If the coating and strip 19 preparation are method steps provided in step-by-step instructions, the strip/cap 19 and 20 will be place and screwed tightly on a bottle containing the block solution above.

Remove strip 19 from blocking solution and do not wash. The strip 19 is now ready to be exposed to the Ebola antigen(s) or virus. Optimally, the strips will be prepared in advance and the packaging of the prepared strip 19 will be according to optimal stability. It is likely to be in a solution pouch (such as one containing blocking solution (PBS/4% BSA)).

Methods of Detection

The following steps may be used to detect a given antigen using coated strip 19:

Ebola is a virus with surface proteins to which the unconjugated coating antibodies bind. The following steps use individual proteins that can be used in the present disclosure. There are different ways in which the disclosed kits may be used to detect, for example, the Ebola protein or virus, further described below.

When using patient blood, collect blood in a small empty tube and centrifuge for 3 minutes at maximum speed on table-top centrifuge if available. In order to avoid the use of a centrifuge (i.e., allowing for even more portability away from hospitals and labs), let blood coagulate at room temperature for at least 10 minutes. Once the blood is clotted, the serum will be free of red blood cells and the prepared strip 19 can be placed in the serum and the capillary action coats the strip with serum (and if the Ebola virus is present in the serum, the virus will bind to the antibodies that are coated on the strip 19). It is desirable, and in most cases necessary, to remove the red blood cells in order to maintain a white background of the strip 19 (for detection purposes).

Washing steps below will go as follows: First remove the strip/cap 19 and 20 from the solution bottle in which the strip/cap 19 and 20 are on, then submerge strip 19 in wash solution by using a squeeze bottle containing the wash solution. As mentioned above, the use of a squeeze bottle (also present in some kits of the present disclosure) will avoid contamination of the wash solution that is to be used multiple times and will also minimize viral exposure of uninfected individuals if wash is discarded appropriately (this can be done by also providing a discard container or bottle). To avoid exposure of a healthcare worker to contaminated strips, these washes can either be done by screwing cap/strip 19 and 20 onto a bottle containing the wash solution or by adding wash solution to the strip 19 via a squeeze bottle. The former (the screw-cap method) can lead to wash solution contamination and so will limit the number of kit uses unless multiple wash bottles are provided.

Wash solution will desirably consist of PBS and may contain a low concentration of detergent reagent (i.e., 0.05%) and/or BSA (2%), which can enhance the specificity of virus (e.g., Ebola) detection (further optimization may be used to vary the different concentrations of detergent and BSA or powdered milk). In some embodiments, powdered milk can be used to replace BSA.

Adding Ebola antigen (Ebola proteins) to solution: About 20 μg (microgram) of each protein per ml of PBS/2% BSA (2 g BSA per 100 ml PBS) is utilized. Make enough to submerge the strip. Incubate at room temperature on a rocker for 1 to 2 hours. Note: the time may be adjusted as desired for optimal result with shortest time. Different concentrations of proteins may be used to optimize conditions for potential low viral loads. If there are low viral loads, for example, more antibodies may be needed during the coating process and during the following steps. As part of the kit, the strip will be submerged in saliva or collected serum (after letting the blood clot) of the patient (as described above).

Wash the strip 19 with wash solution by submerging the strip 19 as part of the strip/cap 19 and 20 in provided wash solution. This will be done by either squeeze bottle or by screwing on the strip/cap 19 and 20 onto a bottle containing the wash solution (described above). Gently shaking and/or inverting and/or letting strip 19 sit in wash solution for a period of time may also be necessary (see above description of wash solution). The time may be adjusted during optimization (short time and best result).

Place strip/cap 19 and 20 in diluted conjugated primary anti-Ebola antibody solution bottle. This bottle will contain about 1 μl conjugated anti-Ebola antibody per 1 ml (1 to 1000 dilution) of PBS/2% BSA solution (optimization will include testing different concentrations for best detection). These conjugated anti-Ebola antibodies are the same anti-Ebola antibodies used during strip 19 coating (see coating antibodies strip preparation portion of protocol); however are conjugated either to horseradish peroxidase (HRP), colloidal gold or biotin. Incubate strip in solution by swishing solution around (or gently shaking) at room temperature for 1 to 2 hrs. Note: Detection may be optimized for various conditions such as sensitivity and duration of exposure.

Conjugated primary anti-Ebola antibodies are commercially available from Abcam (1 Kendall Square, Suite B2304 Cambridge, Mass. 02139-1517), and other companies that also offer the anti-Ebola antibodies. Further, companies such as Nanoprobe (Nanoprobes, Inc., 95 Horseblock Road, Unit 1, Yaphank, N.Y. 11980-2301 USA) can conjugate antibodies to materials such as colloidal gold, and companies such as Thermo Scientific (Thermo Scientific, 3747 N Meridian Rd, Rockford, Ill. USA 61101) can custom label antibodies with HRP and biotin.

(a) HRP-conjugated anti-Ebola antibodies: After submerging strip 19 in a dilution of HRP-conjugated anti-Ebola antibodies and incubation, wash strip 19 with wash solution (as previous described: squeeze bottle or screw-cap method or other). Then, submerge strip 19 in the HRP substrate (undiluted 3,3′,5,5′-tetramethylbenzidine (TMB) solution), by screwing on the strip/cap 19 and 20 onto the bottle containing this TMB solution and incubating for a few minutes. The development should be immediate but can go up to about 8 minutes. Once blue color appears (visualized) and is at the darkness desired, wash strip with tap water.

(b) Colloidal gold-conjugated anti-Ebola antibodies: After submerging strip 19 in diluted colloidal gold conjugated anti-Ebola antibodies, watch development (visualized with a color). Note: different gold particle sizes may be used, as well as silver enhancements if so desired. Additional steps (such as silver enhancement) may be included which may necessitate additional washes (as described above or other) and additional bottles containing appropriate solutions for this method of development.

(c) Biotin-conjugated anti-Ebola antibodies (for enhancement of signal if it is weak): After submerging strip 19 in biotinylated anti-Ebola antibodies and washing (as described previously), add strip/cap 19 and 20 by screwing it onto a bottle containing a solution of streptavidin conjugated with either HRP (see part a) or with colloidal gold (see part b) in PBS/2% BSA solution at a concentration of about 1 μl of streptavidin-conjugate in 5 ml of PBS/2% BSA solution. Incubate for 1 hr (swirl around or gently shake). This additional step can be used to enhance a signal, which may be necessary for low viral loads. The incubation times and concentrations and solution will be adjusted as part of optimization.

This assay can be adjusted to optimize various conditions such as sensitivity, time, concentrations and method of wash (screw-cap or squeeze bottle or other), etc. A few of these optimization examples have been included at each step description; however, variables to optimize are not limited to these examples. Various techniques can be used to visualize a bound antibody (i.e., an antibody that is bound to an Ebola virus or antigen present on a strip). In addition there are different ways of enhancing the above signal. Optimization of the above described methods of detection aims to minimize reagent usage (to allow for more tests per kits), to heighten visual signal if a given virus, such as Ebola, is present, to heighten specificity of a given virus detection, such as Ebola, and to shorten time of assay.

User-friendly tube-transfer (screw-cap method described above): In order to use minimal equipment, each wash, primary solution (i.e., unconjugated coating antibodies or conjugated coating antibodies), detection antibodies, can all be in individual tubes numbered in the appropriate order. Remove caps of all these tubes and a cap 20 with a strip 19 going through it can be placed and screwed on tightly on one tube at a time then transferred from tube to tube 18 in the appropriate order (labeled accordingly). This provides a user-friendly kit and avoids the use of any equipment (avoiding errors and contamination as well as making the kit portable).

If the strips 19 are not prepared in advance, tubes may be provided for this preparation: e.g., one tube with coating antibody, one tube with blocking solution (PBS and BSA or milk, and/or detergent), one tube of PBS alone.

For detection: one tube containing anti-Ebola antibodies conjugated with either HRP, colloidal gold, or biotin, and one tube with wash solution (see above for description). If using HRP, one tube with TMB solution will be needed (and optionally one tube of water). If using colloidal gold, tube(s) for optimal development will be provided. If using biotin, an additional tube of streptavidin conjugated with either HRP or colloidal gold will desirably be provided and any additional tubes needed for development as necessary (see above).

The examples above generally describe a sandwich type assay, but other types of assays are contemplated. In another embodiment, it is contemplated that a sample suspected of containing a virus particle may be placed on the substrate, e.g., a nitrocellulose surface. This surface is then optionally exposed to Bovine Serum Albumin and washed. The area on the substrate surface where the sample was applied is then exposed to a solution of antibodies that bind a molecule on the surface of the virus particle under conditions to provide an area on the substrate with the virus particle bound with an antibody. The antibody may contain a detectable marker such as an antibody conjugated to a fluorescent moiety, dye, quantum dot, or the antibody may be further detected with a second antibody that bind to the primary antibodies constant region, e.g., Fc, or other portions CH2 or CH3 domains of the immunoglobulin. In certain embodiments, the disclosure contemplates that the detectable marker is an enzyme that reacts with compound under conditions such that the compound is converted to visible color or provides a fluorescent signal. For example, the presence of the enzyme horseradish peroxidase (HRP) may be made visible using a compound that, when oxidized by HRP using hydrogen peroxide as the oxidizing agent, yields a characteristic change that is detectable by spectrophotometric methods. HRP catalyzes the conversion of chromogenic substrates (e.g., TMB, DAB, ABTS) into colored products, and produces light when acting on chemiluminescent substrates.

The use of REDS is not limited to Ebola, but to any applicable virus (by replacing the antigen and antibodies in the procedure with that of the applicable virus). This process of detection, including the use of test strips pre-bound with antibodies against the antigen paired with the use of HRP, colloidal gold, or Biotin allows for early detection for a variety of pathogens.

The virus-detection kits of the present disclosure and methods of making and using virus-detection kits of the present disclosure are further described in the embodiments below.

### Example 1

The method may be performed with the following steps:

A blood sample or a standard such as 20 micrograms (ug) of Ebola recombinant protein (recombinant EBOV GPdTM-IBT Bioservices cat# 0501-015) per milliliter (ml) in a total volume of 500 ul are added to nitrocellulose membranes (cut into small strips and placed into Eppendorf tubes). These tubes are then placed on a rocker at room temperature (RT) for about 4 hours.

The strips are were then washed with Phosphate Buffered Saline 3 times (about 1 ml and inverted a few times).

The strips are then blocked by adding 500 ul to 1 ml of 2% Bovine Serum Albumin (BSA) in Wash Buffer (0.05% Tween in PBS), and placed on rocker for about 1.

Strips are washed 3 times with Wash Buffer (about 1 ml each wash and inverted).

Primary antibody are then added: Rabbit anti-EBOV GP pAb (IBT Bioservices cat # 0301-015) are added at 1:500 dilution in 500 ul volume of Wash Buffer. Eppendorf tubes are added on rocker for about 1 hour at RT.

Strips are washed 3 times with Wash Buffer (about 1 ml each wash and inverted). Secondary antibody is then added: Donkey anti-Rabbit IgG (H+L) Cross Adsorbed conjugated HRP Polyclonal (Thermo Scientific Pierce cat#SA1200) is then added at 1:1000 in total volume of 500 ul. Eppendorf tubes are then added on rocker and incubated for about 1 hour at RT.

Strips are washed 3 times with Wash Buffer (about 1 ml each wash and inverted). Strips are then laid out on a plastic surface and TMB substrate for ELISPOT (Fisher cat# NC0304605) are then added in a drop-wise manner to cover the strips.

Results can be detected visually.

Ebola is a virus with surface proteins including Glycoprotein (GP) are used above. This method may be substituted by using colloidal gold labeled conjugated anti-Ebola polyclonal antibodies, which would allow for only one antibody step and wash (no secondary antibody needed). The colloidal gold can be seen without a substrate, which would reduce the extra substrate step.

The present disclosure is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present disclosure and/or the scope of the appended claims.

Further, it should be understood that the herein-described virus-detection kits and/or methods may comprise, consist essentially of, or consist of any of the herein-described components, features and steps, as shown in the figures with or without any feature(s) not shown in the figures. In other words, in some embodiments, the virus-detection kits and/or methods of the present disclosure do not have any additional features other than those shown in the figures, and such additional features, not shown in the figures, are specifically excluded from the virus-detection kits and/or methods. In other embodiments, the virus-detection kits and/or methods of the present disclosure do have one or more additional features that are not shown in the figures.

What is claimed is:

1. A virus-detection kit, said kit comprising:
one or more test strips, each test strip comprising a strip substrate;
one or more reagent solutions in individual solution containers, said one or more reagent solutions comprising (i) at least one wash solution comprising a phosphate buffered saline (PBS) solution, (ii) at least one unconjugated coating antibody solution, and (iii) at least one conjugated primary antibody solution;
one or more test tubes sized to contain a desired amount of a given solution; and
one or more test tube caps sized to seal a given test tube.

2. A virus-detection kit, said kit comprising:
one or more coated test strips, each coated test strip comprising a strip substrate and a coating thereon, said coating comprising at least one unconjugated coating antibody capable of binding with a given virus;
one or more reagent solutions in individual solution containers, said one or more reagent solutions comprising (i) at least one wash solution comprising a phosphate buffered saline (PBS) solution, and (ii) at least one conjugated primary antibody solution;
one or more test tubes sized to contain up to about 1000 milliliters of a given solution; and
one or more test tube caps sized to seal a given test tube.

3. The virus-detection kit of claim 1, wherein said at least one conjugated primary antibody solution comprises at least one of three separate conjugated primary antibody solutions, said three separate conjugated primary antibody solutions comprising (i) a first conjugated primary antibody comprising antibody conjugated with horseradish peroxidase (HRP), (ii) a second conjugated primary antibody comprising antibody conjugated with colloidal gold, and (iii) a third conjugated primary antibody comprising antibody conjugated with biotin.

4. The virus-detection kit of claims 1, wherein said kit further comprises a container sized to house one or more kit components.

5. The virus-detection kit of claims 1, wherein said one or more kit components comprise said one or more test strips, said one or more reagent solutions in said individual solution containers, said one or more test tubes; and said one or more test tube caps.

6. The virus-detection kit of claim 4, wherein said container comprises a sealable box.

7. The virus-detection kit of any one of claims 4, wherein said container comprises an interior container substrate, said interior container substrate having two or more circular storage compartments therein.

8. The virus-detection kit of claim 7, wherein said two or more circular storage compartments comprise ordered locations along said interior container substrate, each of said ordered locations representing a test sample stage within a method of using said kit.

9. The virus-detection kit of claim 7, wherein said two or more circular storage compartments comprise ordered locations along said interior container substrate, each of said ordered locations representing (i) a washed test strip sample, (ii) an unconjugated antibody-coated test strip sample, (iii) an infected test strip sample, and (iv) a used test strip sample.

10. The virus-detection kit of any one of claims 7, wherein said two or more circular storage compartments comprise ordered locations along opposite side walls of said container.

11. The virus-detection kit of any one of claims 7, wherein said two or more circular storage compartments comprise ordered locations, each ordered location being labeled with a number.

12. The virus-detection kit of claims 4, wherein said container further comprises step-by-step instructions on an outer surface of said container, said step-by-step instructions enabling use of said kit by a user.

13. The virus-detection kit of claim 12, wherein said step-by-step instructions assign said number with (i) said washed test strip sample, (ii) said unconjugated antibody-coated test strip sample, (iii) said infected test strip sample, and (iv) said used test strip sample.

14. The virus-detection kit of claims 1, wherein each test strip within said one or more test strips is attached to a given test tube cap within said one or more test tube caps so as to form numerous test strip-cap combinations.

15. The virus-detection kit of claims 1, wherein each test tube cap within said one or more test tube caps snugly removably fits onto each test tube within said one or more test tubes.

16. The virus-detection kit of claim 14, wherein each test strip-cap combination advances within said ordered locations along said interior container substrate as the test strip-cap combination advances thru steps of using said kit.

17. The virus-detection kit of claim 16, said steps of using said kit being provided within said step-by-step instructions.

18. The virus-detection kit of any one of claims 1, said at least one unconjugated coating antibody capable of binding with a given virus, said given virus comprising Ebola.

19. A method of making the virus-detection kit of claims 1.

20. A method of using the virus-detection kit of claims 1.
